(19) United States
(12) Patent Application Publication
(10) Pub. No.: US 2011/0313021 A1
(43) Pub. Date: Dec. 22, 2011

(54) Method to rapidly identify critical p53 target genes that can be utilized for therapeutic intervention

(76) Inventor: Jack Zilfou, Allentown, PA (US)

(21) Appl. No.: 13/134,871

(22) Filed: Jun. 20, 2011

Related U.S. Application Data
(60) Provisional application No. 61/397,906, filed on Jun. 19, 2010.
(60) Provisional application No. 61/397,905, filed on Jun. 19, 2010.

(51) Int. Cl. A61K 31/7088; A61P 35/00; C40B 30/00

(52) U.S. Cl. 514/44 A; 506/7

(57) ABSTRACT

The majority of human cancers inactivate the p53 tumor suppressor network, and consequently, p53 is one of the most studied proteins in cancer biology. Peering into the intricacies of the p53 network should provide insight into the etiology of cancer, which may lead to the identification of molecular targets for therapeutic intervention. p53 carries out its tumor suppressor functions by inducing potent biological outputs, such as programmed cell death, in cells destined for becoming cancerous. p53 is a transcription factor that can regulate expression of numerous genes. The present invention discloses a novel method of rapidly determining the critical p53 target genes that can be utilized for therapeutic intervention. Additionally, this invention discloses suppression of MCAK as a treatment of human cancers and other diseases where p53 levels are elevated.

### CROSS-REFERENCE TO RELATED APPLICATIONS

The present application claims priority from U.S. Provisional Application No. 61/397,905 filed Jun. 19, 2010, and U.S. Provisional Application No. 61/397,906 filed Jun. 19, 2010, both of which are hereby incorporated by reference in their entirety herein.

### FIELD OF THE INVENTION

The present invention, is related to combining various technologies, including RNA interference and microarrays, to query the p53 tumor suppressor network.

### BACKGROUND OF THE INVENTION

Over 1.3 million new cases of cancer are diagnosed annually in the United States, and they are responsible for over 500,000 deaths (National Cancer Institute, see www.cancer.gov). The majority of human cancers abrogate the p53 tumor suppressor network (Hollstein et al., Science 253(5015):49-53 (1991); Yee and Vousden, Carcinogenesis 26(8):1317-1322 (2005)). Consequently, p53 is one of the most studied proteins in cancer biology, presumably because of the belief that insights into its mode of action will produce insights into the etiology of cancer, thus leading to the identification of key molecular targets for therapeutic intervention. p53 carries out its tumor-suppressor functions by inducing potent biological outputs, such as apoptosis or permanent growth arrest, in cells destined for cellular transformation (tumorigenesis) (Zilfou and Lowe, Cold Spring Harb. Perspect. Biol. 1(5):a001883 (2009)). The pathway through which p53 induces apoptosis is quite complex; p53 functions as a sequence-specific transcription factor, and transactivates numerous pro-apoptotic genes, and represses several pro-survival genes (Riley et al., Nat. Rev. Mol. Cell Biol. 9(5):402-412 (2008)). Additionally, p53 has transcription-independent functions (Dumont et al., Nat. Genet. 33(3):357-365 (2003); Moll et al., Curr. Opin. Cell Biol. 17(6):631-636 (2005)). It is generally accepted that the apoptosis function of p53 is an integral component of its tumor suppressor function (Manfredi, Mol. Cell 11(3):552-554 (2003)). Disruption of the apoptotic function of p53 in vivo, either by ablating p53 (Hemann et al., Nat. Genet. 33(3):396-400 (2003)) or by overexpressing the anti-apoptotic gene bcl2 (Schmitt et al., Cancer Cell 1(3):289-298 (2002)), results in significant acceleration of tumors. Moreover, tumors that overexpress the apoptosis antagonist bcl2 show a defective p53 apoptotic program, yet remained sensitive to p53-mediated growth arrest. These findings support the idea that the apoptotic program of p53 is the crucial tumor suppressor function of p53 in those systems.

Whereas several downstream components of the p53-mediated apoptosis pathway have been delineated, to date, many of the key ‘nodes’ of this network have not been elucidated. The identification of critical mediators of p53-dependent apoptosis is the next defining imperative of tumor biology, as such progress will likely yield novel targets for drug discovery. The present inventor developed an “RNA interference” cell-based method to rapidly identify novel, potentially ‘druggable’, and critical effectors of the p53 tumor suppressor network. RNA interference, commonly referred to as RNAi, is a system within living cells that can be engaged to potently and acutely suppress specific gene expression (reviewed in Sharp, Genes Dev. 13(2):139-141 (1999); Fire, Trends Genet. 15(9):358-363 (1999)). The present method, developed in Murine Embryo Fibroblasts (MEFs) was based on the inventor's previous findings that a very low level of p53 (residual p53), attained by the use of RNAi, is sufficient to mediate DNA damage-mediated apoptosis to a similar degree as wild type p53 (WT p53) (Zilfou et al., manuscript in prep). Importantly, the present inventor found that WT p53 and residual p53 transcriptionally regulate unique, yet overlapping, sets of target genes. The present inventor reasoned that genes, specifically encoding for enzymes, that are equally repressed by WT p53 and residual p53 represent putative therapeutic targets, particularly in p53-deficient tumors.

One gene discovered using the aforementioned method and discussed herein is the mitotic centromere-associated kinesin (MCAK, aka kif2c), a p53-repressed kinesin that may serve as an effective therapeutic drug target, particularly in p53-deficient tumors. Kinesins are molecular motors that convert the chemical energy of ATP into mechanical work, particularly on microtubules (MTs) (Hirokawa and Takemura, Exp. Cell Res. 301(1):50-59 (2004)). Kinesins are divided into 14 families (Kinesin-1 to Kinesin-14) comprising approximately 30 different kinesin proteins. Members of this protein family contain four different domains: a conserved catalytic MT-stimulated ATPase domain; a class-specific neck domain necessary for motility; a stalk domain that may be involved in dimerization; and a tail domain that binds to cargo and may regulate activity. MCAK belongs to the Kinesin-13 family. This family is unique in their function at the MTs. Although the majority of kinesins function by ‘walking’ along MTs carrying various cargos such as proteins, chromosomes, and vesicles, members of the Kinesin-13 family catalytically induce MT depolymerization at both ends of a MT and are essential to regulate MT dynamics (Helenius et al., Nature 441(7089):115-119 (2006)). MCAK has a central catalytic domain downstream of a positively charged neck domain. The catalytic and neck domains are sufficient for MT depolymerization, with particular importance reported on the role the neck domain in MT depolymerization.

MCAK was first discovered as a kinesin that associates with mitotic centromeres/kinetochores in cells (Wordeman and Mitchison, J. Cell Biol. 128(1-2):95-104 (1995)). It has been also detected on the spindle poles and in the cytoplasm, where it has been reported to regulate MT dynamics in interphase and mitosis. MCAK has been implicated in the proper alignment of chromosomes at the metaphase plate, and shown to be required for proper chromosome segregation. Displacement of endogenous MCAK from the centromeres by a dominant negative MCAK (MCAK with deleted motor domain) results in a lagging chromosome phenotype during anaphase chromosome movement (Maney et al., J. Cell Biol. 142(3):787-801 (1998)). Additionally, depletion of MCAK in cell extracts and in cells results in a perturbation of MT dynamics and an increase in MT polymer and disruption of proper spindle assembly. These loss-of-function phenotypes support the premise that disruption of MCAK will cause mitotic catastrophe in proliferating cells. There are no reports in the literature of high throughput screens (HTS) aimed at identifying small molecule inhibitors of MCAK. However, one group has identified sulfoquinovosylacyiglycerols (SQAGs), which were originally believed to inhibit mammalian DNA polymerases, as compounds that bind to the neck domain of MCAK (Aoki et al., Febs J. 272(9):2132-2140 (2005)). SQAGs are natural products found in sea urchins, sea algae, and higher plants which have been reported to have antiviral activity, and possibly to inhibit the P-selectin receptor. These compounds are effective at arresting cells in both S phase and M phase, possibly by virtue of their interaction with MCAK. Although SQAGs highlight the use of MCAK inhibitors for cancer therapy, clearly the pleiotropic nature of their action limits their therapeutic potential.

The present inventor optimized a high throughput assay (HTA) for MCAK function and utilized it to screen various libraries of small molecules and natural products for inhibitors of MCAK activity. An inhibitor of MCAK disclosed herein is the natural product gossypol, which was found to inhibit MCAK with a 50% inhibitory concentration (IC50) of approximately 11 μM.

A Major shortcoming in the p53 field is the lack of effective methods to identify the important genes within the p53 tumor suppressor network (a network comprising hundreds and possibly thousands of genes) that can be utilized for therapeutic intervention. Accordingly, it is one object of this invention to provide a tractable method of rapidly identifying critical genes in the p53 tumor suppressor network that may be utilized for therapeutic intervention in a myriad of human diseases. It is also an object of this invention to disclose the inhibition of MCAK as a treatment for cancers with compromised p53. Other objects will appear hereinafter.

### SUMMARY OF THE INVENTION

As a testament to the significance in human disease (particularly cancer) and complexity of the p53 network, there are over 50,000 papers published relating to the biochemical and biological properties of p53. Consequently, there is an ever-expanding list of p53 target genes and effector functions. Still lacking, however, is a meaningful method to rapidly identify and prioritize the critical p53 genes that underpin p53 tumor suppressor functions. The present invention is intended to address this unmet need in the p53 field by disclosing an RNAi-based method in Murine Embryo Fibroblasts (MEFs) to rapidly identify critical p53 target genes involved in the p53-mediated cell death. This method is based on the inventor's observation that a very small amount of p53 (residual p53) can induce p53-mediated cell death in the MEFs stably expressing oncogenes (MEFs stably expressing certain oncogenes undergo p53-dependent cell death, see Lowe et al., Cell 74(6):957-967 (1993)). The present inventor found that residual p53 regulates a unique set of genes compared to WT p53. The common genes regulated by both WT and residual p53 represent the critical subset of genes important for p53-mediated cell death. The genes in this subset are prioritized by selecting p53-repressed enzymes, whereby said enzymes are overexpressed in human tumors lacking functional p53. Other p53 targets that are equally modulated by WT and residual p53 may also represent key target genes for therapeutic intervention.

MCAK represents an example of an enzyme repressed by both WT p53 and residual p53, and thus is probably an important component of the p53 network. It was found that p53 represses MCAK expression in MEFs and various human cancer cell lines, and that expression levels of MCAK correlate to p53 status (whereby cells with high levels of MCAK will often be either deficient for p53 or contain a compromised p53 gene). This finding demonstrates that the critical p53 target genes found in the MEFs are also important in human cells.

The present invention discloses that suppression of MCAK in p53-deficient cells, which overexpress MCAK, can cause selective cell death of said cells. Said suppression can occur via utilization of RNAi techniques to reduce expression of MCAK gene, or via utilization of an inhibitor of the MCAK protein.

MCAK is one example of the numerous critical genes discovered or can be discovered utilizing this method. These genes represent critical genes for therapeutic intervention to treat a variety of human diseases. An example is cancer, a disease characterized by uncontrolled cell growth, whereby said cells are deficient in p53 function most of the time.

### BRIEF DESCRIPTION OF THE FIGURES AND TABLES

FIG. 1 illustrates a ‘heat map’ of the expression levels of various genes in WT MEFs expressing vector, shp53D or shp53E. Genes equally repressed by WT and residual p53 are indicated.

Tab. 1 depicts the expression level of selected genes in the p53 network found in FIG. 1. Data shown in shp53D and shp53E columns are normalized to the corresponding data in the vector column.

FIG. 2 depicts RNA levels, via qPCR analysis, of p53, MCAK and p21 in WT MEFs expressing vector, shp53D, and shp53E. Also shown are RNA levels of these genes in p53 null MEFs.

FIG. 3 is a Western Blot showing the p53, MCAK and actin protein levels in MEFs expressing vector and shp53E in the presence or absence of adriamycin (ADR).

FIG. 4A depicts RNA levels, via qPCR analysis, of p53, MCAK and p21 in H1299 cells harboring a doxycycline(Dox)-inducible p53, and treated with the indicated doses of Dox.

FIG. 4B is a Western Blot showing p53, MCAK, p21 and actin protein levels in the cells described in FIG. 4A.

FIG. 4C depicts RNA levels, via qPCR analysis, of MCAK in the human breast cancer cells MCF7 (which have WT p53) and T47D (which have mutant p53) in the presence or absence of ADR.

FIG. 4D is a Western Blot showing p53, MCAK and actin protein levels in the cells and treatments described in FIG. 4C.

FIG. 5A depicts RNA levels, via qPCR analysis, of MCAK in the indicated human lung cancer cell lines with the indicated p53 status.

FIG. 5B depicts RNA levels, via qPCR analysis, of MCAK in the indicated human kidney cancer cell lines with the indicated p53 status.

FIG. 6A depicts RNA levels, via qPCR analysis, of p21 and MCAK in MEFs expressing shp53E in the presence or absence of small interfering RNAs (siRNAs) targeting MCAK (siMCAK).

FIG. 6B depicts a colony formation assay for WT MEFs expressing vector or shp53E in the presence of either a siControl or siMCAK.

FIG. 6C shows the quantification of the colony formation assay in FIG. 6B.

FIG. 7A depicts RNA levels, via qPCR analysis, of MCAK levels in WT MEFs expressing either vector or the indicated short hairpins targeting MCAK (shMKs).

FIG. 7B depicts RNA levels, via qPCR analysis, of MCAK levels in p53 null MEFs expressing either vector or the indicated short hairpins targeting MCAK (shMKs).

FIG. 7C shows the survival of the MEFs described in FIG. 7A in response to the indicated doses of ADR.

FIG. 7D shows the survival of the MEFs described in FIG. 7B in response to the indicated doses of ADR.

FIG. 8A shows the absorbance at 650 nm in the MCAK ATPase assay as inorganic phosphate levels are increased.

FIG. 8B shows the absorbance at 650 nm in the MCAK ATPase assay as MCAK protein levels are increased.

FIG. 9 illustrates the chemical structure of gossypol.

FIG. 10A shows the activity of MCAK in the MCAK ATPase assay in response to the indicated doses of gossypol.

FIG. 10B illustrates the effects of gossypol on the ability of MCAK to depolymerize microtubules in an in vitro microtubule assembly assay.

FIG. 10C shows the quantification of the data presented in FIG. 10B.

FIG. 10D depicts the effects of the indicated doses of gossypol on the survival of WT and p53 null MEFs.

### DETAILED DESCRIPTION OF THE INVENTION

The present invention provides a method to rapidly identify critical genes regulated by the p53 tumor suppressor network. Said method compares the ‘gene expression profiles’ in at least three distinct populations of ‘intentionally altered’ ‘cells’. As used herein, ‘cells’ refers to the functional basic unit of life, and may refer to any mammalian cells, including but not limited to, mouse cells, rat cells, monkey cells, and human cells. Cells of non-mammalian origin may also be used, providing they can contain the p53 gene and can undergo p53-dependent cell death. As used herein, intentionally altered describes the introduction of DNA sequences to engage the RNA interference machinery of the cell to suppress expression of a desired gene in the cell. RNA interference, or RNAi, refers to a system within living cells that can regulate gene expression and function (reviewed in Sharp, Genes Dev. 13(2):139-141 (1999); Fire, Trends Genet. 15(9):358-363 (1999)). Additionally, as used herein, ‘gene expression profiles’ refers to the aggregate of the expression products (referred to as Ribonucleic acids or RNAs) of all genes within a cell. Gene expression profiles can be determined using a myriad of techniques available in the field, including but not limited to, DNA ‘microarrays’ (may be referred to as gene chips, DNA chips, or biochip). As used herein, ‘microarrays’ refer to a collection of microscopic DNA spots attached to a solid surface, that can be used measure expression levels of large numbers of genes simultaneously, thus providing the user with a gene expression profile.

The p53 tumor suppressor can regulate many different genes within a network (p53 tumor suppressor network) that can cause very potent biological responses in cells, including cell death (see above for details). Wild-type p53 (WT p53), as used herein, refers to the normal amount of p53 found in cells. The present invention is based on the finding that very small amounts of p53, referred to herein as residual p53, can cause p53 dependent cell death in cells. Of significance, residual p53 can still regulate a set of genes within cells that is unique, but overlaps with the set of genes regulated by WT p53. The present invention focuses on the prioritization of said genes regulated by both WT p53 and residual p53.

In one embodiment of this method, the cells utilized were murine embryo fibroblasts (MEFs). Three groups of MEFs were altered to yield three unique populations of MEFs. Said MEFs were altered by the introduction of retroviral vectors comprising short-hairpin RNA sequences targeting the p53 gene (shRNAs), or empty vector (referring to vector void of any sequences targeting the p53 gene). Two unique shRNA vectors targeting p53 were previously generated, referred to as shp53D and shp53E, which are capable of reducing p53 gene expression and protein levels to varying amounts (Hemann et al., Nat. Genet. 33(3):396-400 (2003); Dickins et al., Nat. Genet. 37(11):1289-1295 (2005)). Retroviral transduction of shp53D and shp53E into MEFs results in a reduction of p53 protein by approximately 95% and 99%, respectively (Zilfou et al., manuscript in preparation), while the MEFs transduced with empty vector contain 100% p53 (WT p53). The ability of the remaining p53 to cause cell death in these MEFs was evaluated in the presence of the oncogenes E1A or E1A plus ras (E1A/ras). Said oncogenes introduced in MEFs comprise a well-established system to study p53-dependent cell death (Lowe et al., Cell 74(6):957-967 (1993)). In this system, the MEFs containing WT p53 (empty vector) and residual p53 (shp53D) underwent p53-dependent cell death in response to DNA damage (the DNA-damaging drug adriamycin was used). In contrast, MEFs containing shp53E did not undergo p53-dependent cell death in response to the same doses of adriamycin. Additionally, MEFs containing empty vector (WT p53), shp53D (residual p53) or shp53E have unique, yet overlapping, gene expression profiles (Zilfou et al., manuscript in prep).

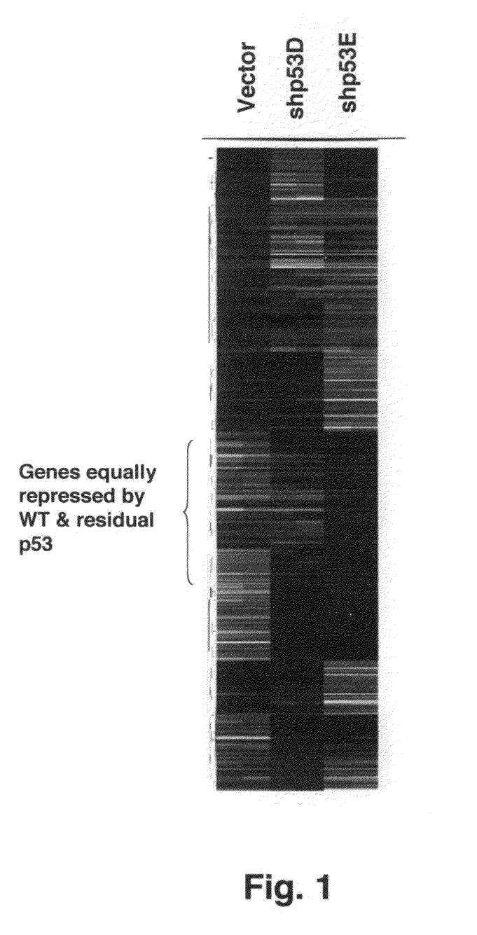

The present inventor reasoned that determining the overlap between the gene expression profiles of MEFs containing WT p53 and residual p53 under conditions that cause p53-dependent cell death will yield critical genes in the p53 network involved in cell death. Accordingly, the three groups of MEFs described above were generated, and treated with 0.5 milligrams per milliliter adriamycin for four (4) hours and gene expression profiles were determined using microarrays covering >30,000 genes of the mouse genome. FIG. 1 depicts the gene expression data, whereby each line within the figure represents a particular gene. As expected, a significant reduction in the expression of the p53 gene as well as a reduction in previously identified p53 activated genes, such as p21^{waf1/cip1} (p21) and Mdm2, in the MEFs harboring shp53D and shp53E (Tab. 1). Additionally, we found a marked increase in expression of previously identified p53-repressed genes, such as stathmin, in these MEFs (Tab. 1). Standard techniques for extracting RNA for microarray analysis can be utilized, and any type of microarray may be utilized.

The aforementioned microarray experiments identified the critical genes within the p53 network for p53-dependent cell death. Additionally, this method lead to the discovery of several novel p53 target genes that are regulated by both residual p53 and WT p53. The present invention also includes a prioritization scheme intended to provide the critical genes for therapeutic intervention. Said scheme is based on the following criteria: 1) genes that are equally repressed by WT and residual p53; 2) ‘bonafide’ p53 repressed genes; 3) genes whose products are potentially ‘druggable’ (i.e. have enzymatic activity); 4) genes whose products play a role in p53-dependent cell death (inhibition of gene function leads to cell death); 5) genes that are upregulated in human tumors lacking functional p53; 6) availability of an intellectual property niche. The term ‘bonafide’, as used herein, refers to previously described criteria of p53 target genes (Riley et al., Nat. Rev. Mol. Cell Biol. 9(5):402-412 (2008)).

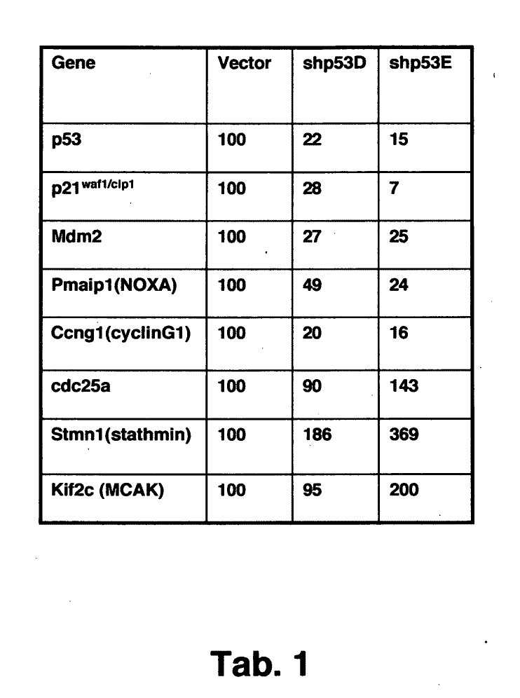

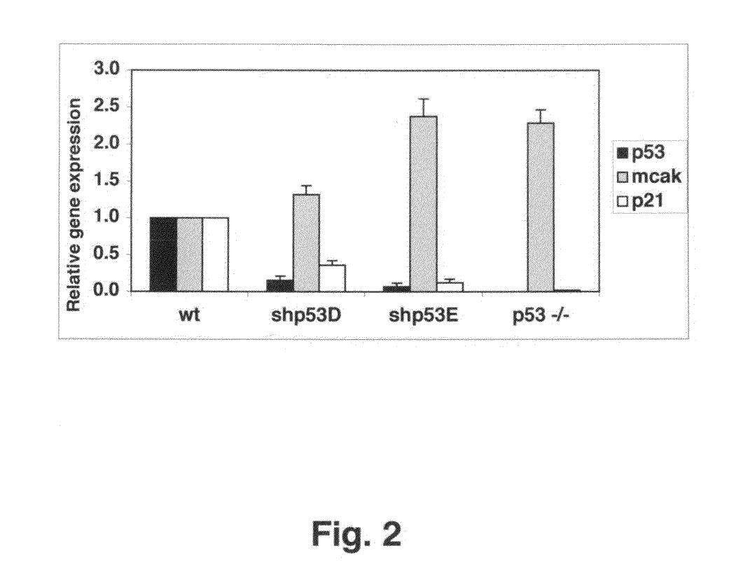

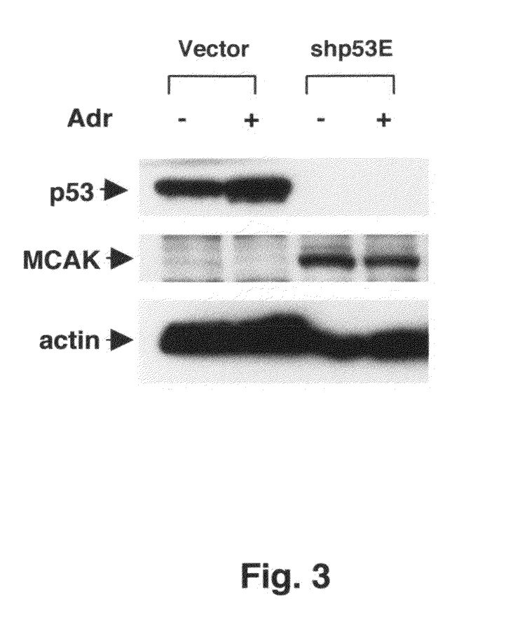

As an example, said prioritization scheme has identified mitotic centromere-associated kinesin (MCAK, aka kif2c) as gene that fits these criteria. The unique role of MCAK in mitotic checkpoints and in p53-dependent cell death renders it a unique drug target. In separate validation experiments, MCAK repressed by WT and residual p53. As depicted in FIG. 2, expression of MCAK in p53 null MEFs (deficient for p53) and shp53E MEFs is markedly higher than in WT MEFs and shp53D MEFs, as assessed by quantitative polymerase chain reaction (qPCR) (standard technique utilized to determine levels of specific RNA in cells). As expected, expression of the p53 transactivated gene p21^{waf1/cip1} (p21) is notably reduced in p53 null MEFs, shp53D, and shp53E MEFs compared to WT MEFs containing vector. Additionally, p53 represses MCAK protein. FIG. 3 shows a Western Blot for p53 and MCAK in WT MEFs expressing vector or shp53E in the presence or absence of the DNA damaging agent adriamycin (ADR) (ADR increases levels of p53), and demonstrates that ablation of p53 in the shp53E MEFs results in a marked increase in MCAK protein levels. The protein actin is shown as a control to demonstrate equal amounts of overall protein in each treatment group.

The present invention also provides for the suppression of MCAK as a treatment for various diseases that are implicated in p53 deficiency. Specifically, human cancers that have a non-functional p53 can be treated by suppressing MCAK expression. Some evidence of this can be gleaned from the experiments carried out and discussed below.

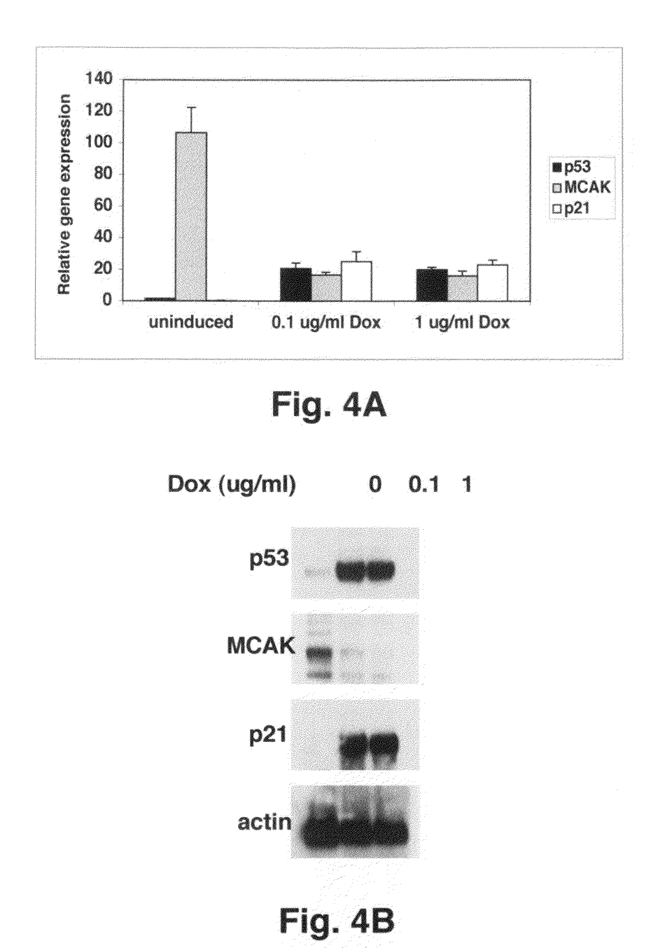

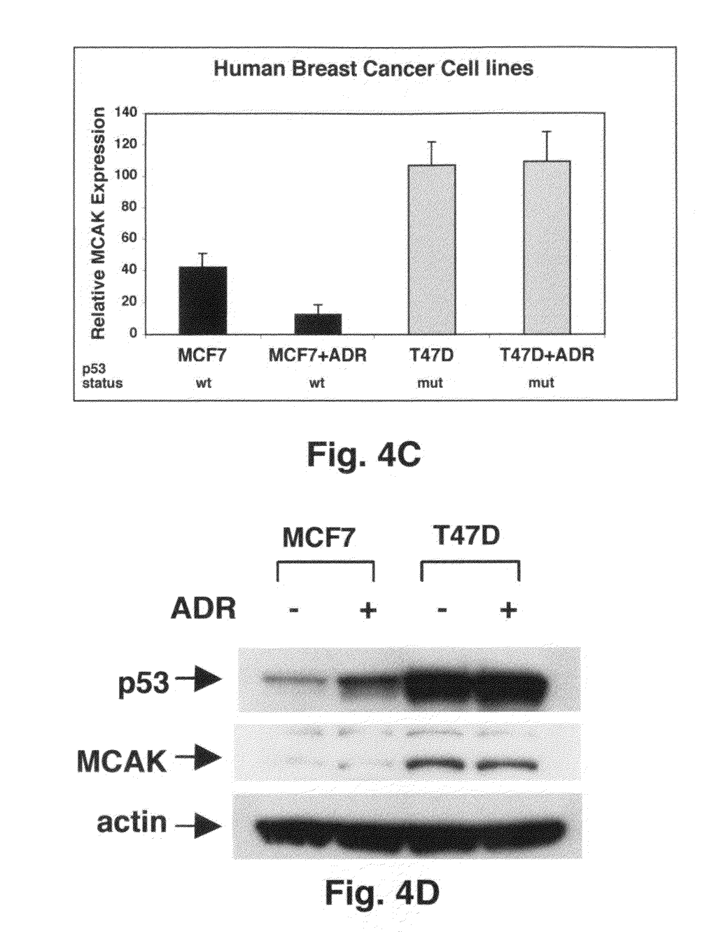

Several different cell lines were utilized to determine the ability of p53 to repress MCAK protein and RNA expression. The H1299 human lung cancer cell line was engineered to harbor a doxycycline (Dox)-inducible p53. The addition of 0.1 or 1 μg Dox/ml significantly increases p53 RNA and protein expression (FIGS. 4A and 4B). Consistent with MCAK being a p53-repressed gene, the increase in p53 level results in significant decrease in the expression of MCAK RNA and protein levels (FIGS. 4A and 4B). In contrast, the RNA and protein levels of p21, a p53 transactivated gene, markedly increased in response to Dox treatment (FIGS. 4A and 4B). Actin is shown as a loading control (FIG. 4B).

The breast cancer cell lines MCF7 and T47D were also utilized, containing endogenous WT and mutant p53, respectively. MCF7 cells have a notably lower level of MCAK RNA and protein than found in the T47D cells (FIG. 4C). These data suggest that uninduced WT p53, not mutant p53, represses MCAK levels. We next induced p53 levels by treating the cells with the DNA damaging drug, adriamycin (ADR). As depicted in FIG. 4D, treatment with ADR significantly increased WT p53 protein levels in MCF7 cells, but did not affect mutant p53 levels in the T47D cells. Induction of WT p53 in MCF7 cells results in a marked decrease in MCAK RNA (FIG. 4C). No change in MCAK RNA levels was found between the treated and untreated T47D cells (FIG. 4C). Of note, mutant p53 (as found in T47D) has been shown to be more stable than WT p53 because of its compromised ability to transactivate the MDM2 gene, which negatively regulates p53 protein.

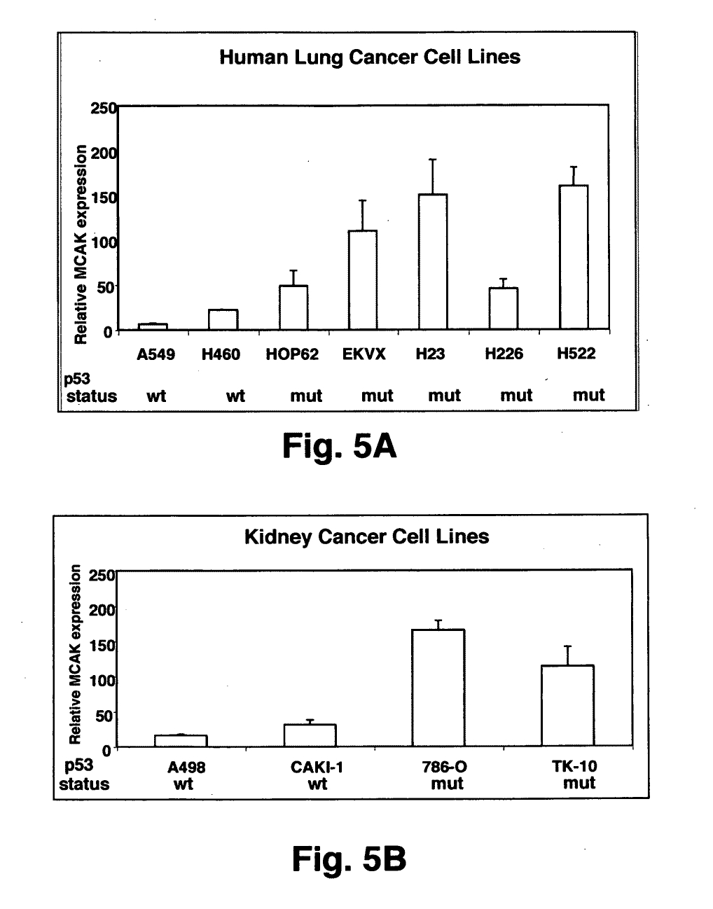

Additionally, mutation of p53 correlates with high MCAK levels in numerous human cancer cells lines. FIG. 5 shows qPCR analysis of MCAK levels for human lung (FIG. 5A) and kidney cancer cell lines (FIG. 5B) with differing p53 status. Cell lines harboring mutations in p53 consistently have higher levels of MCAK expression compared to cells with WT p53. This difference in MCAK levels may provide the selectivity for potential therapeutic intervention.

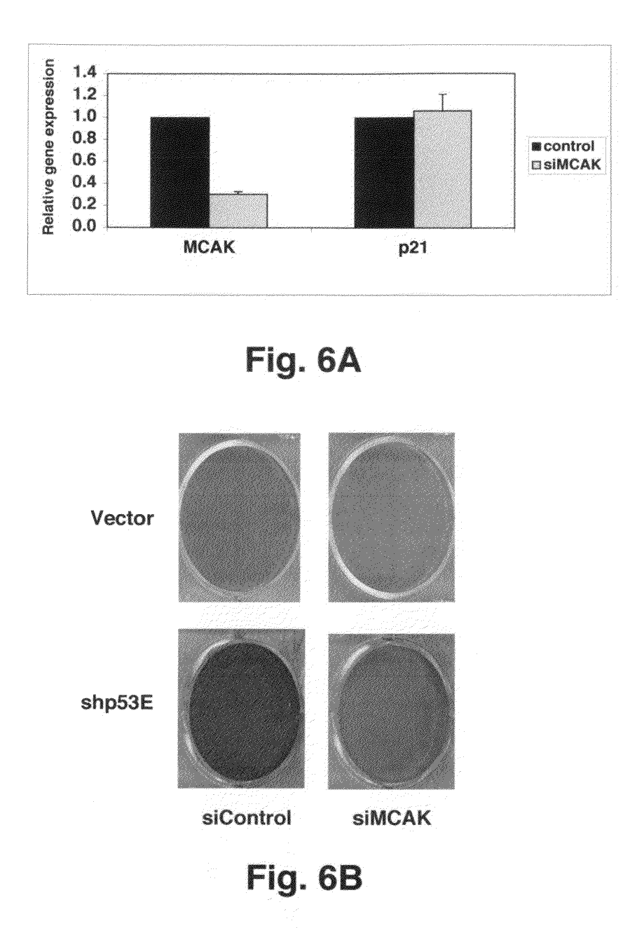

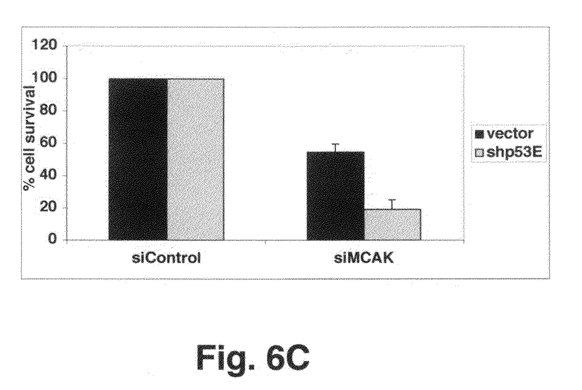

The p53 network imparts potent effects on cell growth and survival. Consequently, we sought to determine the effects of acute suppression of MCAK, a putative p53-repressed gene, on these cellular properties in WT MEFs transduced with vector or shp53E. The suppression of p53 by shp53E in MEFs results in a significant increase in MCAK expression (FIG. 2 and FIG. 3). Initially, MEFs with empty vector were transduced and shp53E MEFs with ‘pooled’ small interfering RNAs targeting murine MCAK (siMCAK). siMCAK effectively suppressed MCAK expression in these MEFs (FIG. 6A). In contrast, siMCAK had relatively no effect on expression of the p21 gene (FIG. 6A).

Colony-formation assays were performed using the MEFs treated with siMCAK to determine the effects of acute suppression of MCAK on cell growth. In this assay, p53 deficiency results in a greatly enhanced ability of untransformed cells to form colonies when plated at clonogenic density. We have previously reported the ability of shp53E to enhance growth of MEFs in a colony formation assay (Dickins et al., Nat. Genet. 37(11):1289-1295 (2005)). As depicted in FIG. 6B, siMCAK markedly decreases colony formation vector and shp53E MEFs. However, quantification of these data reveals that while siMCAK resulted in an approximate 47% decrease in colonies in WT MEFs, the same treatment resulted in an approximate 81% decrease in colonies (FIG. 6C). These data reveal greater sensitivity of shp53E MEFs to loss of MCAK than vector MEFs. Acute suppression of MCAK using siRNAs targeting MCAK has been previously shown to decrease growth of human cancer cell lines (Shimo et al., Cancer Sci. 99(1):62-70 (2008)).

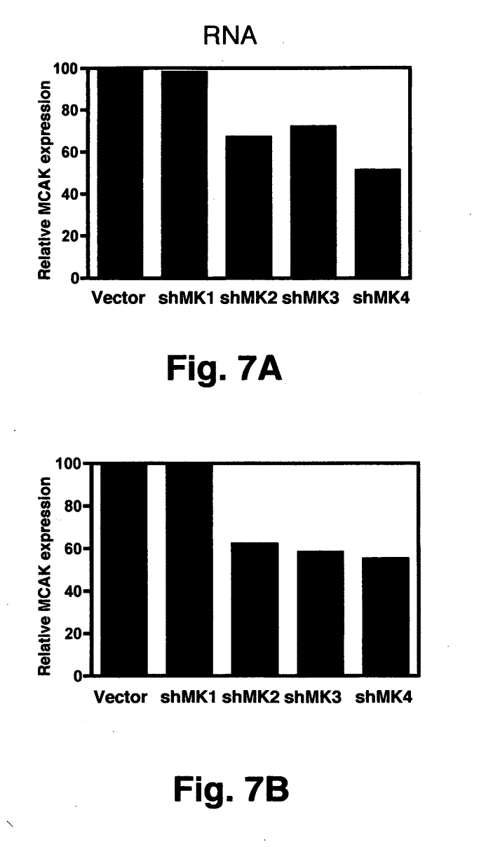

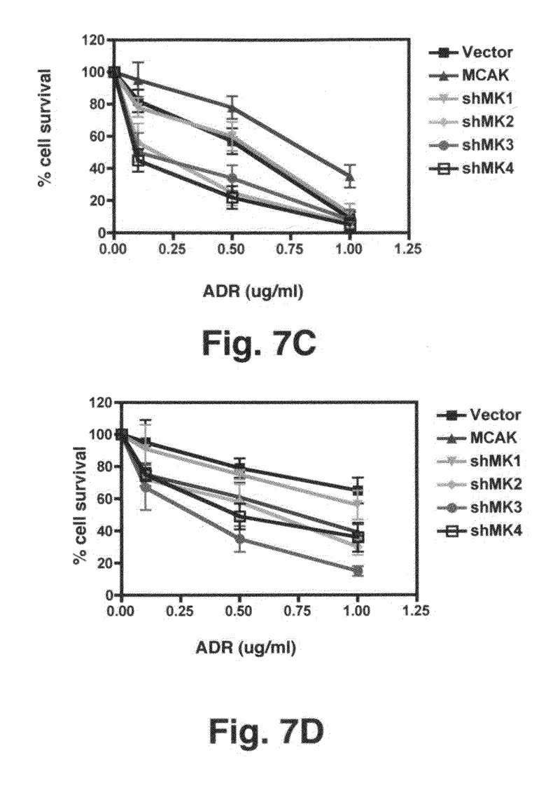

MEFs expressing the E1A or the E1A/RAS oncogenes become sensitized to p53-mediated cell death after DNA damage. To investigate the effects of stable suppression of MCAK expression on p53-mediated cell death, several short hairpin RNAs (shRNAs) targeting distinct sequences in the murine MCAK gene (shMK1, shMK2, shMK3, shMK4) were generated and cloned them into a retroviral expression vector. Said shRNAs were subsequently tested for their activity after introduction into WT and p53 null MEFs stably expressing E1A/ras. Evaluation of shMKs in these MEFs revealed that shMK2, shMK3, and shMK4 moderately reduced MCAK expression, while shMK1 had relatively no effect on MCAK expression (FIGS. 7A and 7B). It was found that introduction of shMK2, shMK3 and shMK4 in WT or p53 null MEFs (expressing E1A/ras) markedly sensitized cells to ADR treatment (FIGS. 7C and 7D). Significantly, introduction of shMK1, the shRNA not affecting MCAK expression, into WT and p53 null MEFs had no effect on sensitivity of these cells to ADR (FIGS. 7C and 7D). Additionally, MCAK was transduced into WT and p53 null MEFs stably expressing E1A/ras and subsequently these cells were treated with various doses of ADR. Consistent with a role in modulating p53-mediated cell death, introduction of MCAK into WT MEFs resulted in relative resistance to ADR when compared to vector MEFs (FIG. 7C). Interestingly, enforced expression of MCAK in p53 null cells sensitized them to ADR (FIG. 7D). Of note, p53 null MEFs are markedly more resistant to ADR treatment than WT MEFs in the presence of E1A/ras.

Disruption of the mitotic checkpoint has been shown to be an effective approach to cancer treatment. Anti-mitotic agents, such as vinca alkaloids and taxanes, which interfere with microtubule (MT) dynamics by targeting tubulin and MTs, are widely used and efficacious in the clinic. However, MTs on only play a critical role in mitosis, as they are also required for other important cellular functions, such as intracellular transport. Consequently, the anti-mitotic agents act on both proliferating and post-mitotic cells, leading to numerous adverse effects, including neuropathy and myeloesuppression (Jackson et al., Nat. Rev. Cancer 7(2):107-117 (2007)). Therefore, therapeutics that target the mitotic spindle by novel mechanisms of action and provide greater specificity for tumor cells represent a ‘rational’ path to treat cancer. At least one member of the kinesin superfamily of MT motor proteins may fill this therapeutic niche. Specifically, KSP (Eg5/kinesin-5) is a mitotic kinesin that is required for proper centrosome separation and the formation of a bipolar spindle during mitosis. Inhibitors of KSP have been identified and characterized. Prolonged exposure of cells to these inhibitors has been shown to induce apoptosis after mitotic arrest (Tao et al., Cancer Cell 8(1):49-59 (2005)). Moreover, inhibitors of KSP have potent anti-tumor activity in human tumor xenograft models (Sakowicz et al., Cancer Res. 64(9):3276-3280 (2004)); and have recently entered clinical trials for cancer therapy.

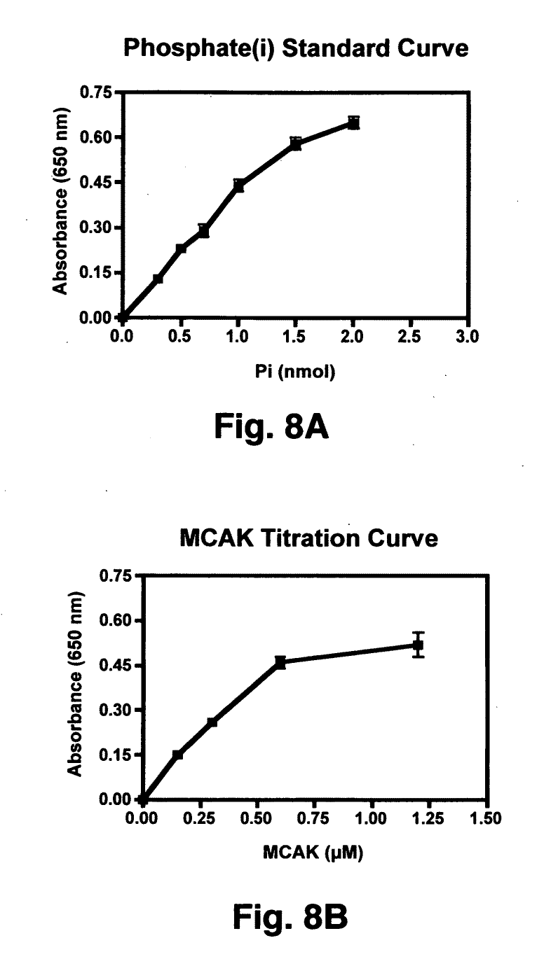

Based on the instant data supporting a role for MCAK in p53 mediated cell death, it can be reasoned that MCAK will also fill a therapeutic niche for rational drug design targeting the mitotic checkpoint. Importantly, cancer cells with abrogated p53 contain high levels of MCAK, thereby providing a genetic determinant for therapeutic use of MCAK inhibitors. Accordingly, the inventor sought to identify and characterize inhibitors of MCAK function. MCAK utilizes the energy of Adenosine triphosphate (ATP) hydrolysis to carry out its MT depolymerization function. The inventor exploited this characteristic to utilize and optimize a commercially available in vitro assay that measures MCAK ATPase activity (Cytoskeleton, Denver Colo.). The components of the assay are: recombinant MCAK protein, ATP, pre-formed MTs, detection reagent for inorganic phosphate (Pi), and the appropriate buffers for the reaction. We utilized a glutathione S-transferase (GST)-tagged recombinant MCAK protein corresponding to amino acids 182 to 646 of the human MCAK protein, a region of the protein that contains the kinesin motor domain of MCAK. The ATPase activity of MCAK directly correlates to the presence of Pi in the reaction mixture. The components of this assay were titrated and optimized, and the data are shown in FIGS. 8A and 8B.

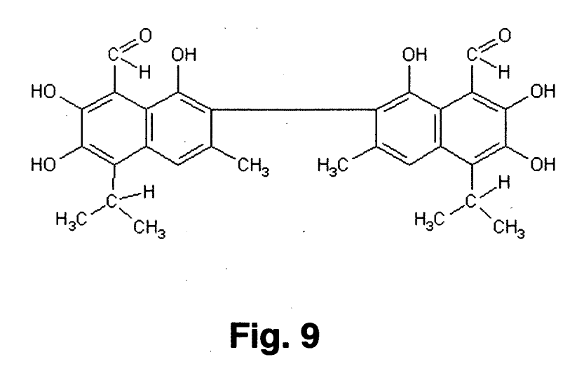

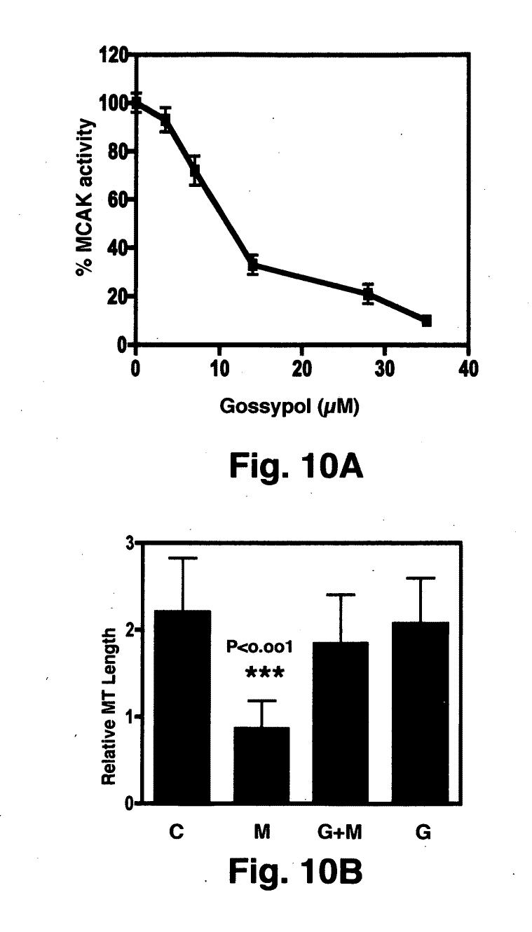

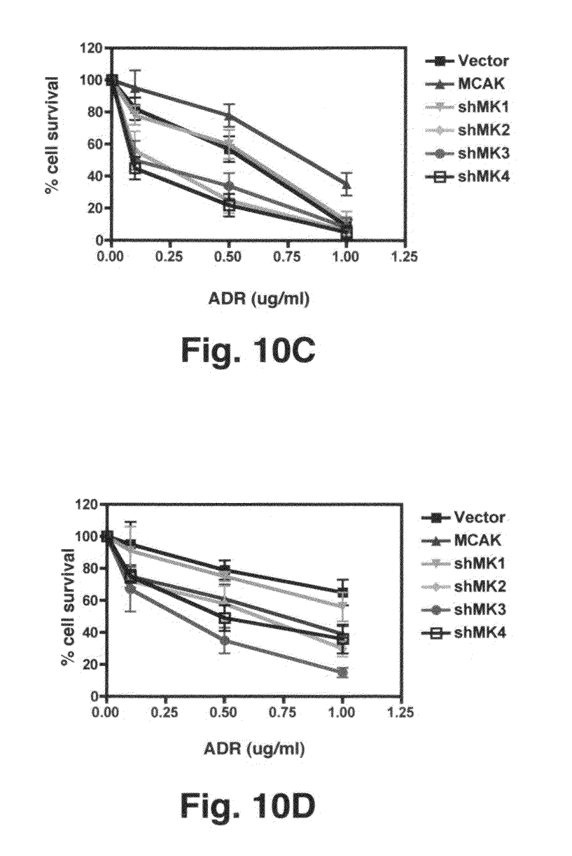

The inventor used the MCAK ATPase to carry out a high-throughput screen (HTS) of a library of synthetic small molecules and various natural products. While the inventor will not report on the small molecule MCAK inhibitors in this disclosure, the inventor will discuss gossypol, a natural product that we discovered is a potent inhibitor of MCAK activity. Gossypol is a polyphenol derived from the cotton plant (Gossypium malvaceae), and its chemical structure is shown in FIG. 9. FIG. 10A depicts a dose-response curve for gossypol in the MCAK activity assay. Gossypol inhibits MCAK activity with a 50% inhibitory concentration (IC50) of approximately 11 μM. Since MCAK has been shown to depolymerize MTs (Helenius et al., Nature 441(7089):115-119 (2006)), the inventor sought to determine the effects of gossypol on this biochemical function. The inventor employed a previously described in vitro MT stabilization assay (Desai et al., Cell 96(1):69-78 (1999)). As expected, introduction of MCAK in this assay depolymerized paclitaxel-stabilized MTs, resulting in markedly shorter MTs compared to control (FIGS. 10B and 10C). Importantly, the addition of gossypol inhibited the ability of MCAK to depolymerize MTs, while gossypol alone did not affect the MT lengths (FIGS. 10B and 10C). The inventor quantified these results by measuring the length of at least 200 different MT strands for each treatment (FIG. 10C).

To investigate the effects of gossypol on cell survival, the inventor treated numerous cell lines with various doses of gossypol. The inventor found that p53 null MEFs, which contain high levels of MCAK, are more sensitive to gossypol than WT MEFs (FIG. 10D). This is a significant finding, as p53 null cells are generally more difficult to kill. We are currently testing the effects of gossypol on different cancer cell lines with varying p53 status.

MCAK is presented merely as an example of a critical gene in the p53 network identified using the present invention. Many other genes identified may serve as viable therapeutic targets. Additionally, the prioritization scheme presented to rapidly identify critical p53 target genes may be expanded to include genes transactivated by both WT and residual p53, as well as genes differentially regulated (either repressed or transactivated) by INT and residual p53.

In other embodiments, different cell lines may be utilized, including but not limited to human fibroblast cells, instead of MEFs, whereby shRNAs targeting the particular p53 found in those cells.

1. A method to rapidly identify critical genes in the p53 tumor suppressor network comprising:
a population of cells;
said cells containing material to suppress p53 expression in said cells;
treatment of said cells with agents to induce cell death;
comparison of gene expression profiles of said populations of cells containing differing amounts of p53 levels;
overlap between gene expression profiles of population of said cells with intact p53 and population of said cells with reduced p53 levels yields important genes in p53 network.

2. The method recited in claim 1, wherein a population of cells comprises any type of mammalian cell.

3. The method recited in claim 1, wherein a population of cells comprises non-mammalian cells.

4. A method of treating disease by suppression of the MCAK or KIF2C gene product in cells with increased levels of the p53 gene product.

5. The method recited in claim 4, wherein said disease is human cancer.

6. The method recited in claim 4, wherein said disease has cells with high levels of p53 gene product.
